(19) United States
(12) Patent Application Publication
(10) Pub. No.: US 2008/0195158 A1
(43) Pub. Date: Aug. 14, 2008

(54) Spinal Fusion Device

(76) Inventor: Malan De Villiers, Irene (ZA)

Correspondence Address: LADAS & PARRY LLP, 224 SOUTH MICHIGAN AVENUE, SUITE 1600, CHICAGO, IL, 60604, US

(21) Appl. No.: 11/911,001

(22) PCT Filed: Apr. 11, 2006
(86) PCT No.: PCT/IB2006/000828

§ 371 Date: Dec. 17, 2007

(30) Foreign Application Priority Data
Apr. 12, 2005 (ZA) 2005/02948

(51) Int. Cl. A61B 17/58; A61B 17/56

(52) U.S. Cl. 606/280; 606/246; 606/301

(57) ABSTRACT

The application concerns a spinal fusion device (26) which can be used to fuse respective vertebrae (L5, S1) relative to one another. The device includes plate sections (10 and 12) which can be fixed to the respective vertebrae. These plate sections are hinged to one another to allow the device to accommodate different geometries at different fusion sites.

### BACKGROUND TO THE INVENTION

THIS invention relates to a spinal fusion device.

Lower back problems are frequently attributable to spinal disc failure between the lower lumbar vertebra (L5) and the sacrum (S1), and often necessitate fusion of these vertebrae. The surgical fusion procedure is carried out anteriorly and involves, firstly, distraction of the vertebrae and insertion between them of a so-called ALIF (anterior lumbar interbody fusion device) and, secondly, rigid connection of the vertebra to one another by means of an anterior lumbar plate.

The most commonly used anterior lumbar plate is the co-called Pyramid plate. This is a rigid metal plate which is fastened to each of the two vertebrae by screws passing through the plate and engaged in predrilled holes in the relevant vertebrae.

The known Pyramid plate has a shape specifically suited to the L5/S1 fusion. However fusion of vertebrae is often required at other spinal levels. The fixed shape of the Pyramid plate may render it geometrically unsuitable for fusion of other vertebrae at such other levels.

### SUMMARY OF THE INVENTION

The present invention provides a spinal fusion device for fusing respective vertebrae relative to one another, the device comprising first and second plate sections which are fixable to the respective vertebrae and which are hinged to one another.

Preferably the plate sections have holes therein to receive fastening screws which are in use inserted through the holes and into the respective vertebrae, thereby to fix the plate sections to the vertebrae. Each plate section may have two such holes in spaced apart relationship, and the holes in at least one of the plate sections may be divergent.

The device can include a guard which is mountable on the plate sections in a position to extend at least partially over the holes, thereby to prevent withdrawal of the fastening screws. Conveniently one plate section has a passage extending therein from an anterior surface thereof, the other plate section has a threaded screw hole extending therein from an anterior surface thereof, one end of the guard is insertable into the passage and the other end of the guard has a hole therein arranged to register with the screw hole when the plate sections are hinged to one another, such that a screw can be passed through the guard hole and threaded into the screw hole to secure the guard anteriorly over the hinged plate sections.

The plate sections may have intercalating projections formed with aligned hole therein to receive a hinge pin which hinges the plate sections to one another.

The plate sections should be hinged to one another at a hinge axis which is displaced anteriorly from the axis of normal pivotal movement between those vertebrae when the plate sections are fixed to the vertebrae, thereby to provide a rigid fusion between the vertebrae.

Other features of the invention are set forth in the appended claims.

### BRIEF DESCRIPTION OF THE DRAWINGS

The invention will now be described in more detail, by way of example only, with reference to the accompanying drawings in which:

FIG. 1 shows an elevation of a lower plate section of a spinal fusion device according to the invention;

FIG. 2 shows a cross-section at the line 2-2 in FIG. 1;

FIG. 3 shows a cross-section at the line 3-3 in FIG. 1;

FIG. 4 shows an elevation of an upper plate section of a spinal fusion device according to the invention;

FIG. 5 shows a cross-section at the line 5-5 in FIG. 4;

FIG. 6 shows a cross-section at the line 6-6 in FIG. 4;

FIG. 7 shows a perspective view of a spinal fusion device according to the invention in an assembled condition;

FIG. 8 shows a side view of the guard used in the assembled device seen in FIG. 7;

FIG. 9 shows a cross-section at the line 9-9 in FIG. 7; and

FIG. 10 diagrammatically illustrates the spinal fusion device in use.

### DESCRIPTION OF THE ILLUSTRATED EMBODIMENT

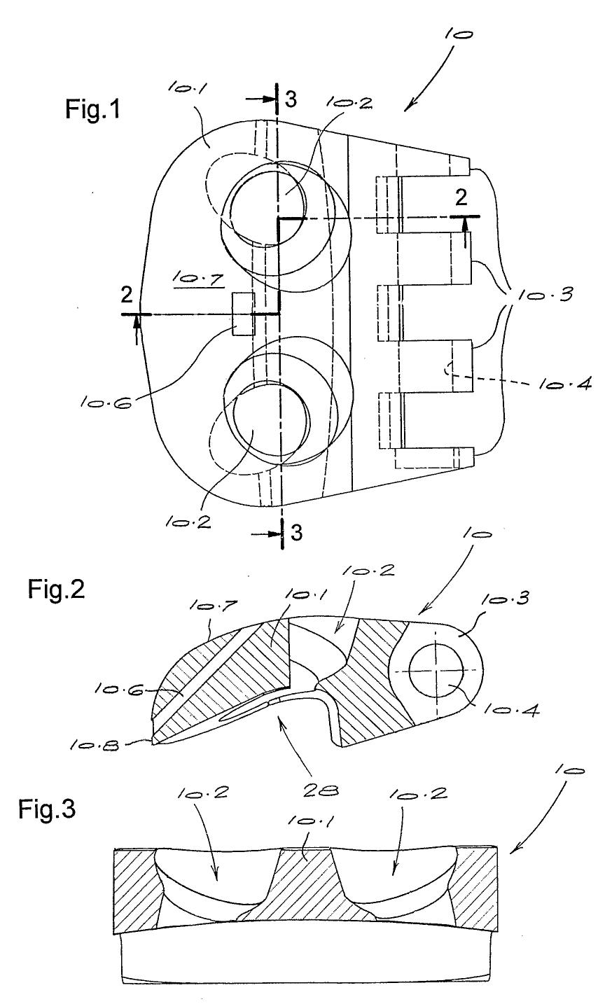

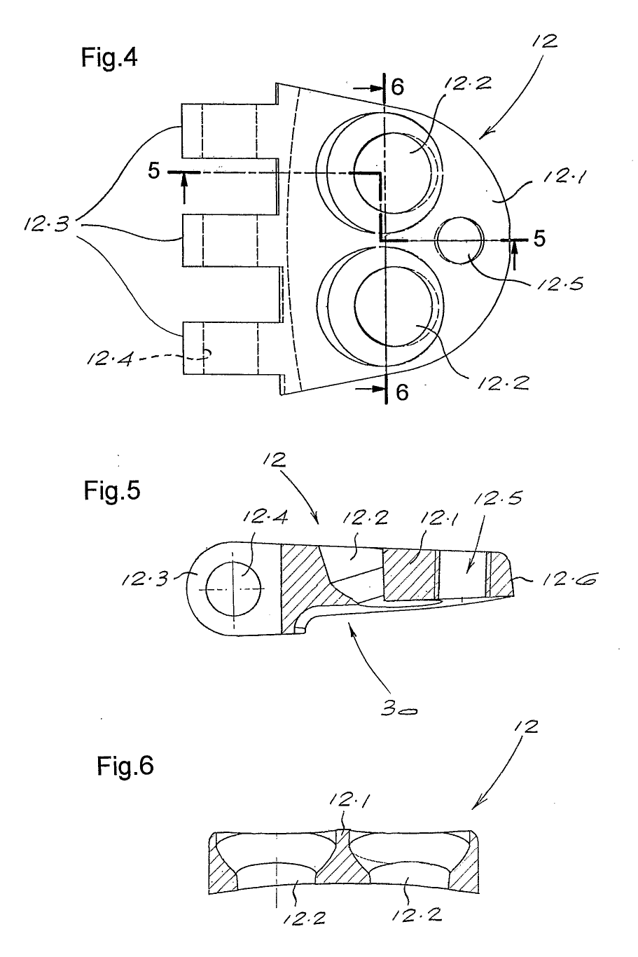

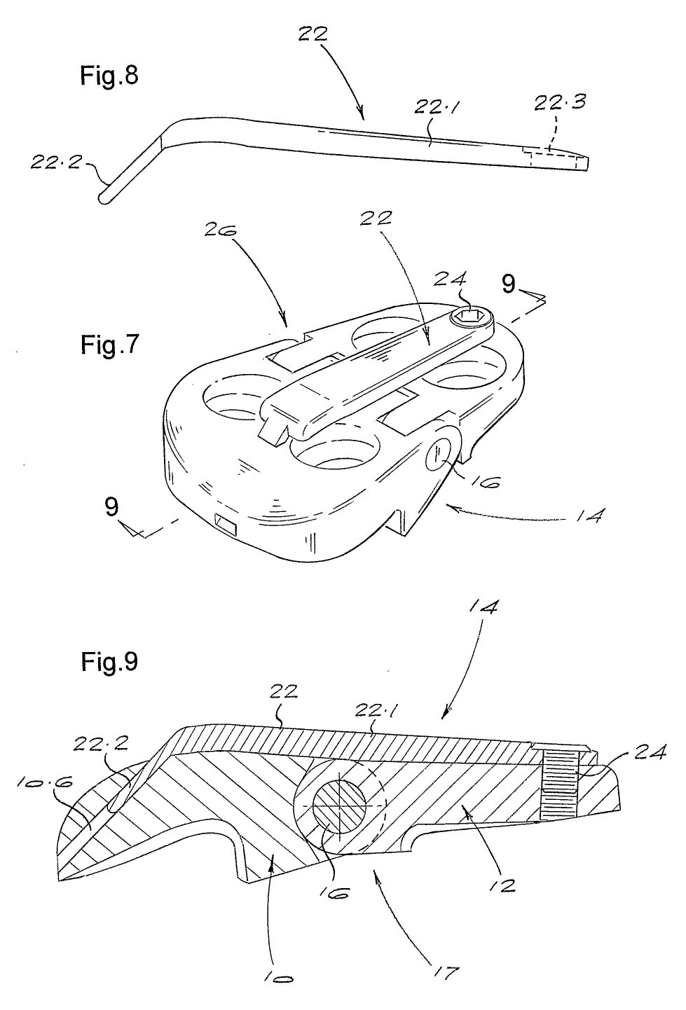

FIGS. 1 to 3 illustrate the lower or bottom plate section 10 and FIGS. 4 to 6 the upper or top plate section 12 of a spinal fusion device 14 according to the present invention. The assembled device 14 is illustrated in FIGS. 7, 9 and 10.

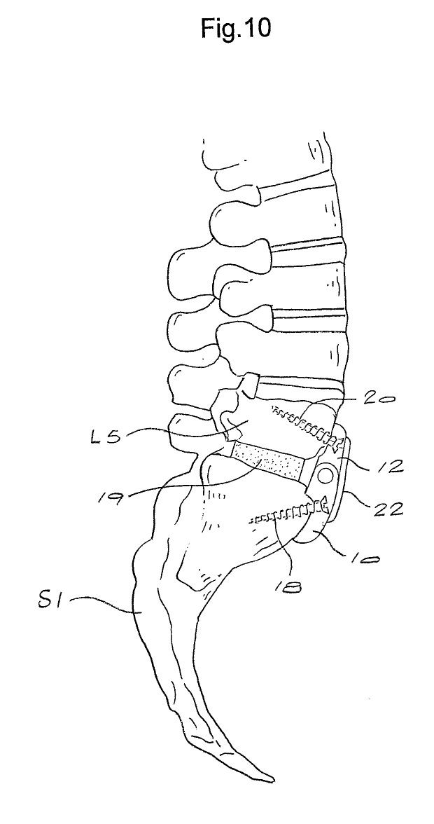

In use, the bottom plate section 10 will be fixed to a lower one of two vertebrae which are to be fused and the top plate section 12 will be fixed to the upper one of the two vertebrae. In a typical lumbar fusion the bottom plate section is fixed to the sacrum S1 and the top plate section is fixed to the lower lumbar vertebra L5, as illustrated in FIG. 10 and described in more detail below.

The bottom plate section 10 is made of grade 5 titanium and consists of a body 10.1 formed with spaced apart, countersunk holes 10.2 that diverge from one another as illustrated. Spaced apart projections in the form of hinge knuckles 10.3 project from what is operatively an upper edge of the plate section and are formed with aligned pin-receiving holes 10.4. The plate section 10 is also formed with a narrow, inclined internal passage 10.6 which proceeds from the anterior surface 10.7 of the body 10.1 to what is operatively a lower edge 10.8 thereof, as illustrated.

The top plate section 12 is made of grade 4 titanium and consists of a body 12.1 formed with spaced apart, countersunk holes 12.2 and with spaced apart projections in the form of hinge knuckles 12.3 projecting from what is operatively a lower edge of the plate section. The knuckles 12.3 are formed with aligned pin-receiving holes 12.4. In addition, the plate section 12 is formed with a threaded hole 12.5 close to what is operatively an upper edge 12.6 of the plate section.

The arrangement of the hinge knuckles 10.3 and 12.3 is such that the knuckles can intercalate with one another, with the pin-receiving holes 10.4 and 12.4 aligned with one another, when the plate sections 10 and 12 are brought together, as shown in FIG. 7, to form a lumbar fusion device 26. A pin 16 passes through the aligned holes and hinges the plate sections relative to one another.

In the lumbar fusion illustrated in FIG. 10, the sacrum S1 and the lower lumbar vertebra L5 are distracted in the normal way and an ALIF 19 is positioned between them. Screws 18 extending through the holes 10.2 of the bottom plate section are then screwed tightly into predrilled holes in the sacrum S1. Similarly, screws 20 extending through the holes 12.2 of the top plate section 12 are screwed tightly into predrilled holes in the lower lumbar vertebra L5. The sacrum and lower lumbar vertebra are accordingly fused.

After insertion and tightening of the screws 18 and 20 a guard 22, seen in FIG. 8, is mounted anteriorly over the assembly of plate sections. The guard 22 is made in one piece of grade 4 titanium and consists of a slender, elongate body 22.1, a locating section 22.2 extending at an angle from one end of the body and a hole 22.3 at the other end of the body. The guard is installed by sliding the relatively narrow locating section 22.2 into the passage 10.6, thereby locating the guard relative to the bottom plate section, and by fastening the opposite end of the body 22.1 relative to the top plate section 12 by means of a screw 24 passing through the hole 22.3 and into the threaded hole 12.5.

In the installed position seen in FIG. 10 the plate sections 10 and 12 form, in combination, a rigid structure, i.e. the device 26 is rigid when installed. Although the plates 10 and 12 are initially freely hinged to one another about the axis of the pin 16, the fact that the axis of normal pivotal movement between the sacrum S1 and the lumbar vertebra L5 is displaced posteriorly from the axis of the pin, together with the fact that the plate sections are fixed to the respective vertebrae and the fact that the screws are constrained by the plate sections, makes pivotal movement between the plate sections impossible.

The guard 22 serves the important function of preventing withdrawal of the screws 18 and 20 from the respective vertebrae. Those skilled in the art will appreciate that it is possible for screws engaged in bone to work loose with passage of time, and that withdrawal of the screws 18 and 20 as a result of such loosening is undesirable, particularly in view of the close proximity of important structures such as the iliac vein which could be damaged by the screws. As will be apparent from FIG. 7, the guard extends at least partially over the heads of the screws 18 and 20, thereby preventing their withdrawal.

The hinging of the plate sections 10 and 12 to one another gives rise to an important advantage of the illustrated device. Those skilled in the art will appreciate that there is sometimes a need to perform a spinal fusion between vertebrae remote from the L5/S1 site. In such situations, the conventional, rigid Pyramid plate is not entirely suitable because it is specifically shaped to suit the geometry at the L5/S1 site while the actual geometry varies from site to site in the spine. The hinged nature of the illustrated embodiment enables the plate sections 10 and 12 to be pivoted relative to one another, before they are fixed to the vertebrae, to enable the assembled plate 26 to conform to a fairly wide range of different geometries at other sites while still achieving effective fusion. The relative slenderness of the guard 22 enables it to bend to some degree to conform to the plate sections over a range of pivotal positions.

It will also be noted that the posterior surfaces of the plate sections 10 and 12 are formed with rebates 28 and 30 respectively which are shaped to allow the plate sections to conform to the shapes of the vertebral bones.

1. A spinal fusion device for fusing respective vertebrae relative to one another, the device comprising first and second plate sections which are fixable to the respective vertebrae and which are hinged to one another.

2. A spinal fusion device according to claim 1 wherein the plate sections have holes therein to receive fastening screws which are in use inserted through the holes and into the respective vertebrae, thereby to fix the plate sections to the vertebrae.

3. A spinal fusion device according to claim 2 wherein each plate section has two spaced apart holes therein to receive fastening screws.

4. A spinal fusion device according to claim 3 wherein the spaced apart holes in at least one of the plate sections are divergent.

5. A spinal fusion device according to claim 2 and including a guard which is mountable on the plate sections in a position to extend at least partially over the holes.

6. A spinal fusion device according to claim 5 wherein one plate section has a passage extending therein from an anterior surface thereof, the other plate section has a threaded screw hole extending therein from an anterior surface thereof, one end of the guard is insertable into the passage and the other end of the guard has a hole therein arranged to register with the screw hole when the plate sections are hinged to one another, such that a screw can be passed through the guard hole and threaded into the screw hole to secure the guard anteriorly over the hinged plate sections.

7. A spinal fusion device according to claim 6 wherein the guard comprises an elongate body which can be bent to accommodate different pivotal positions of the plate sections relative to one another.

8. A spinal fusion device according to claim 1 wherein the plate sections have intercalating projections formed with aligned hole therein to receive a hinge pin which hinges the plate sections to one another.

9. A spinal fusion according to claim 1 wherein the plate sections have posterior surfaces formed with rebates shaped for cooperation with anterior surfaces of the respective vertebrae.

10. A spinal fusion according to claim 1 wherein the plate sections are hinged to one another at a hinge axis which is displaced anteriorly from the axis of normal pivotal movement between those vertebrae when the plate sections are fixed to the vertebrae.
